(19) United States
(12) Patent Application Publication
(10) Pub. No.: US 2015/0021478 A1
(43) Pub. Date: Jan. 22, 2015

(54) NON-DESTRUCTIVE SUGAR CONTENT MEASURING APPARATUS

(71) Applicant: DAESUNG TECH CO., LTD., Gumi-shi (KR)

(72) Inventors: Ho Yun Lee, Daegu-shi (KR); Young Su Do, Daegu-shi (KR); No Jin Park, Seoul (KR)

(73) Assignee: DAESUNG TECH CO., LTD., Gumi-shi (KR)

(21) Appl. No.: 14/070,848

(22) Filed: Nov. 4, 2013

(30) Foreign Application Priority Data
Jul. 22, 2013 (KR) 10-2013-0086161

(51) Int. Cl. G01N 33/02; G06F 17/18; G01N 25/00; G01N 21/35; G01N 21/27

(52) U.S. Cl. CPC G01N 33/025; G01N 21/3563; G01N 21/27; G01N 25/00; G06F 17/18; G01N 2201/126; G01N 2201/125
USPC 250/338.4

(57) ABSTRACT

A non-destructive sugar content measuring apparatus is provided and includes a measuring sensor portion for including a spectral sensor which receives a near infrared ray from the light which is reflected by the flesh FB of the fruit F of which the sugar content is measured, an LED light source which has LEDs circularly arranged, an optical sensor which receives light reflected by a flesh of a fruit F, and a temperature sensor; a casing including a measuring sensor portion and has a panel portion which has a digital display for displaying a brix value as a digital value and operational switches, the panel portion and the operational switches being mounted on a front face thereof; a main circuit board PB for including a Central Processing Unit (CPU) which is embedded in the casing and processes electric signals from the light sensor and performs a calculation and determination

### BACKGROUND OF THE INVENTION

1. Field of the Invention

The present invention relates to a non-destructive sugar content measuring apparatus, and more particularly to an apparatus for measuring sugar content of fresh foods, i.e. fruits, in a non-destructive manner, which has a compact structure with a remarkable improvement of portability.

2. Description of the Prior Art

Conventionally, there have been various attempts of measuring sugar content of a fruit by using a property of light wavelengths when a near infrared ray is emitted to and reflected from the fruit, as a method of measuring sugar content of a fruit in a non-destructive manner.

For example, Korean Patent Laid-Open Publication No. 10-2011-0111970 discloses an integrated light sensor and method for measuring sugar content of fruit, which includes an integrated light sensing module which has a light emitting diode for emitting a light with a wavelength capable of permeating fruit tissue, and a photodiode for detecting the light reflected from the fruit tissue, and a sugar content measuring sensor which has a bundle of light fibers which are disposed on the light emitting diode and the photodiode and are in direct contact with a surface of the fruit, a load cell unit attached to and fixed to a lower end of the bundle of light fibers, a load cell, a light emitting diode chip, and a substrate disposed under a photodiode chip.

In the structure of the above-mentioned Patent Laid-open Publication, the light emitting diode as a light transmitting portion, the photodiode as a light receiving portion, the load cell, and the bundle of light fibers are configured to be arranged on a single chip, and there is provided a sensor for collecting light by using the bundle of the light fibers and the load cell.

However, a structure in that the light emitting diode and the photodiode are arranged as the light transmitting unit and light receiving unit on the single chip has a very complex manufacturing process. Further, since a number of different materials are integrated on the chip, it is difficult to automate a process, thereby requiring manually performing the process. Accordingly, a yield of the process is low and a manufacturing cost increases and makes it difficult to actually apply the process.

Further, since the technology of the Korea Patent Laid-Open Publication discloses only the structure of the sensor, and does not disclose a suitable structure of the entire sugar content measuring apparatus, it is difficult to understand a manner of actually using the sensor. Accordingly, there is an increasing necessity for a sugar content measuring apparatus which a user is capable of carrying, is non-destructive, and can accurately read a numeric value of the sugar content.

### SUMMARY OF THE INVENTION

The present invention has been made to address the above-mentioned problems in the prior art, and an aspect of the present invention is to provide a non-destructive sugar content measuring apparatus, based on a well-known near infrared spectrum method, in which a measuring sensor unit is provided as a sensor which has an improved structure without an extension of a light source because it has a simple structure and a conventional light fiber is used, thereby providing the high-accuracy sugar content measuring apparatus while carrying it simply, so as to reduce a manufacturing cost and to maximize portability.

In accordance with an aspect of the present invention, a non-destructive sugar content measuring apparatus is provided. The apparatus includes: a measuring sensor portion 30 for including an LED light source which has LEDs circularly arranged, an optical sensor which receives light reflected by a flesh of a fruit, and a temperature sensor, in order to predict sugar content through a statistical analysis method in which the optical sensor to be used as a spectral sensor receives a near infrared ray from the light which is emitted from the light source and reflected by the flesh FB of the fruit F of which the sugar content is measured; a casing including a rear half body on which the measuring sensor portion extends outwardly and a front half body combined with the rear half body to constitute the casing of the sugar content measuring apparatus, the front half body having a panel portion which has a digital display for displaying a brix value as a digital value and operational switches, the panel portion and the operational switches being mounted on a front face thereof; a main circuit board for including a Central Processing Unit (CPU) which processes electric signals from the optical sensor and performs a calculation and determination, an EPROM which stores temperature data from the temperature sensor and light source data from the LED light source, and a rechargeable electric power supplying portion, the main circuit board being embedded in the casing; and the CPU enabling the digital display to display the sugar content as a numeric value which is obtained by processing a light waveform, which is measured based on wavelength data corresponding to electric processing signals from the optical sensor, temperature data from the temperature sensor and light source data from the LED light source, by means of a statistic analysis method, wherein the optical sensor is an element on the sensor unit U which is mounted on a circuit board separated from the main circuit board, and has a spectral filter and a CMOS Image Sensor (CIS) in which nano-filter arrays are mass-manufactured and monolithically integrated on a CIS wafer by using a semiconductor process of an optical lithography, a sensor signal processing microcomputer SC is mounted, as a pretreatment means for processing and transmitting an electric signal from the optical sensor to the CPU, on an identical circuit board as that for the optical sensor, and constitutes an independent sensor unit, and the optical sensor is disposed in the measuring sensor portion and exposed outwardly.

The non-destructive sugar content measuring apparatus of the present invention as described above has an improved portability and an increased accuracy of the measurement.

Further, the non-destructive sugar content measuring apparatus is provided as an entire apparatus which is optimized to measure the sugar content and uses an LED light source having a waveband of a single wavelength which is optimized for sugar. Accordingly, it is possible to reduce a manufacturing cost and decrease a consumption of electric power. As described above, the non-destructive sugar content measuring apparatus can be constructed to meet the technical objects of the preceding application prior to the present application, and be independently optimized to have a compact size for portability and distribution.

### BRIEF DESCRIPTION OF THE DRAWINGS

The above and other aspects, features, and advantages of the present invention will be more apparent from the following detailed description taken in conjunction with the accompanying drawings, in which:

FIGS. 1A and 1B are a side view and a plan view illustrating an exemplary structure of a non-destructive sugar content measuring apparatus to which a technology of the present invention is applied;

FIG. 2 is a rear view illustrating a sensor unit of the non-destructive sugar content measuring apparatus to which the technology of the present invention is applied;

FIG. 3 is a plan view illustrating a measuring sensor of the non-destructive sugar content measuring apparatus according to the present invention;

FIG. 4A is a sectional view illustrating a contact mount which can be attached to the measuring sensor of the non-destructive sugar content measuring apparatus according to the present invention;

FIG. 4B is a sectional view illustrating the measuring sensor of the non-destructive sugar content measuring apparatus according to the present invention, taken along a line A-A in FIG. 3;

FIG. 5 is an enlarged sectional view illustrating a contact state of the measuring sensor with a fruit to be measured, which shows a property of a light source employed to the measuring sensor of the non-destructive sugar content measuring apparatus;

FIG. 6 is a perspective view illustrating a sensor unit, which is a circuit board, for receiving a light sensor used in the non-destructive sugar content measuring apparatus according to the present invention;

FIG. 7 is a mimetic diagram illustrating a process of manufacturing the measuring sensor which has a suitable structure to be applied to the non-destructive sugar content measuring apparatus according to the present invention;

FIG. 8 is a mimetic diagram illustrating a sensor of the measuring sensor unit for the non-destructive sugar content measuring apparatus to which the technology of the present invention is applied;

FIG. 9 is a block diagram illustrating an entire hardware of the non-destructive sugar content measuring apparatus according to the present invention;

FIG. 10 is a flowchart illustrating an operation of the sensor measuring unit for the non-destructive sugar content measuring apparatus according to the present invention;

FIG. 11 is a flowchart illustrating an operation of the non-destructive sugar content measuring apparatus to which the technology of the present invention is applied;

FIG. 12 is a graph illustrating optical spectrums of fruits with various sugar contents, in which the optical spectrums are read for a statistical analysis of the non-destructive sugar content measuring apparatus according to the present invention;

FIG. 13 is a graph illustrating an interrelation of the sugar content measured by an optical sensor to an actual sugar content of a fruit to establish a sugar content prediction model in the case of an apple, which is a statistical analysis of the non-destructive sugar content measuring apparatus; and

FIG. 14 is a side view, a plan view and a rear view illustrating a mock-up of the non-destructive sugar content measuring apparatus according to the present invention, which shows a whole structure of the apparatus to be produced.

### DESCRIPTION OF THE EMBODIMENT OF THE PRESENT INVENTION

Hereinafter, a preferred embodiment of the present invention to achieve the object will be described with reference to the accompanying drawings.

The structure and operation of the non-destructive sugar content measuring apparatus 100 according to the present invention will be sequentially described along a flow of an electric signal from a structural member.

[A Hardware Structure of the Sugar Content Measuring Apparatus 100]

The non-destructive sugar content measuring apparatus 100 of the present invention and a mock-up for an actual produce will be described with reference to the accompanying drawings.

It will be understood that a casing of the non-destructive sugar content measuring apparatus 100 of the present invention is formed to have not only an exemplary shape as shown in the drawing but also various shapes through an injection molding.

The casing of the non-destructive sugar content measuring apparatus 100 of the present invention, considering portability, is manufactured as a general half combination which is divided into a front half body 1 and a rear half body 20, and has a main Printed Circuit Board (PCB) embedded therein.

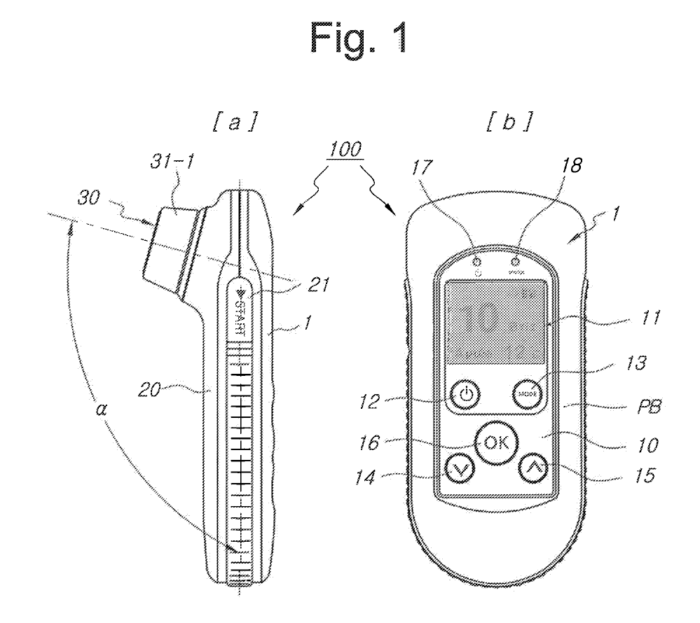

As shown in FIG. 1B, the front half body 1 has a panel portion 10 disposed thereon. The panel portion 10 includes an LCD, preferably a digital display 11, for displaying a value of sugar content (Brix) by a numeric value.

The panel portion 10 includes an electric power switch 12, a mode selection switch 13, up and down switches 14 and 15 for a menu selection, and a conformation switch 16 for conforming a selection, which are made in the form of a membrane type switch and are arranged on the panel portion 10. Further, the panel portion 10 includes an Light Emitting Diode displaying means as an electric power indicating light 17 and a charge state indicating light 18.

The operational switches are selectively included in the non-destructive sugar content measuring apparatus 100. For example, in the case where a non-destructive sugar content measuring apparatus is specialized for a specific fruit, i.e. apple, the up and down switches 14 and 15 for selecting the kind of fruits may be unnecessary.

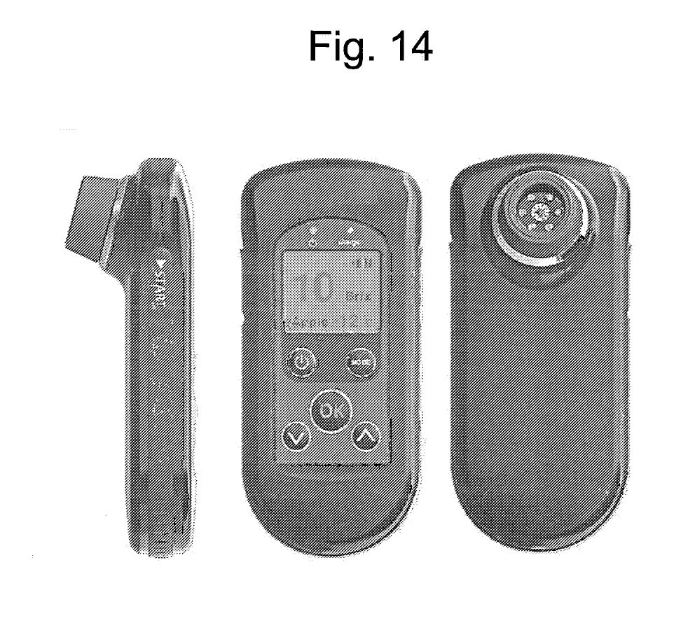

The preferable digital display 11 is constituted of the Liquid Crystal Display which includes a sugar content displaying portion for displaying a value of the sugar content by a Brix value, an object displaying portion for displaying a fruit to be measured, a temperature displaying portion for displaying a temperature around a surrounding environment, and a charge state displaying portion for displaying a state of a battery to which an electric power is charged, which are arranged at preferable positions thereon, as shown in FIGS. 1B and 14.

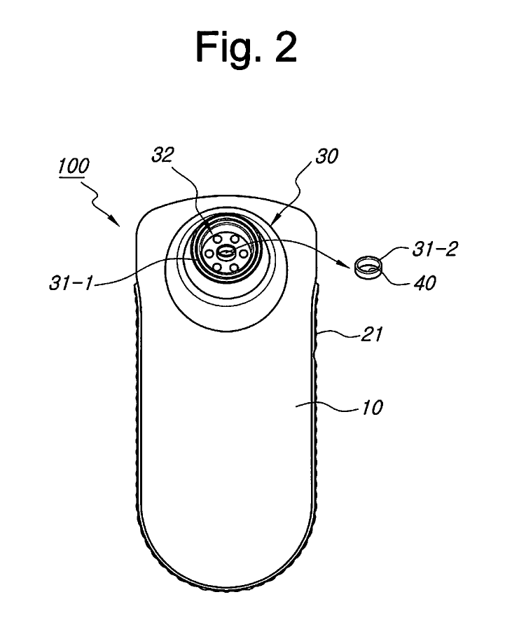

As shown in FIG. 2, the rear half body 20 is combined with the front half body 1 so as to constitute a casing, and includes the main Printed Circuit Board (PB) which is an internal member, and a measuring sensor portion 30.

The rear half body 20 has the measuring sensor portion 30 which is the important structural element and is disposed at an upper portion thereof in a cylindrical shape. On a side portion of the casing constituted of a combination of the front half body 1 and the rear half body 20, a measuring start button 21 is disposed for an operational convenience of a user, which allows the user to grasp the casing with a hand and measure the sugar content of a fruit.

The measuring sensor portion 30 of the cylindrical assembly extending from a surface of the rear half body 20 is preferably arranged to be at a predetermined inclined angle α with a central axis of the casing in order to avoid an interference with the fruit during the measurement.

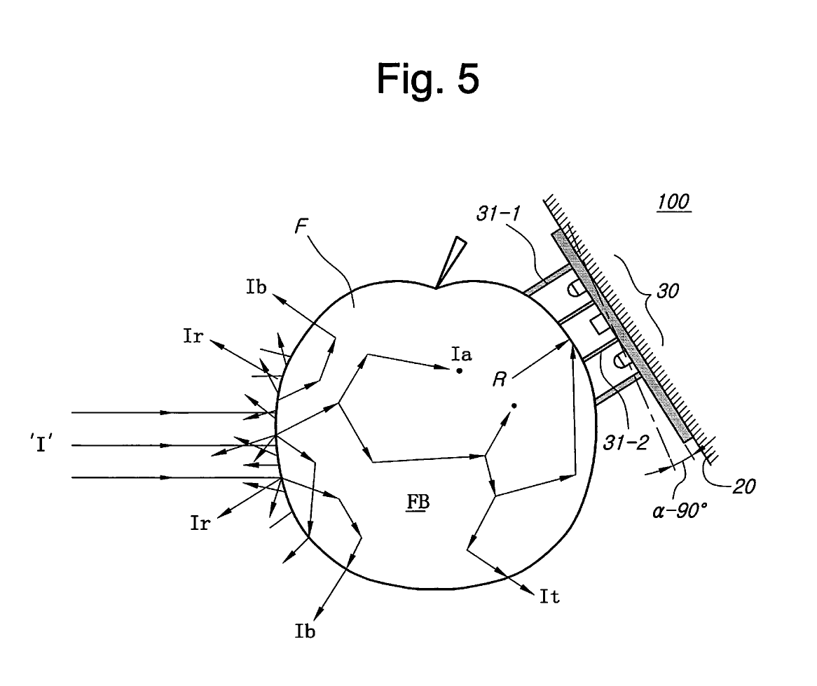

The reason that the measurement starting button 21 is positioned at the side of the casing is to allow the user to operate the measuring starting button 21 with one hand grasping the casing so that the measurement can be started in a state that the non-destructive sugar content measuring apparatus 100 is in contact with the measured fruit F without a sway, as shown at left portion in FIG. 5. Other operational switches are preferably arranged on the panel portion 10 shown in FIG. 1B because they are operated in a state that the non-destructive sugar content measuring apparatus 100 is spaced from the fruit F before or after measuring the sugar content.

The measuring sensor portion 30 extending from the rear surface of the casing has an inclined angle α with a central line of the casing, so as to be in contact with the fruit F such as an apple, which is measured, at a contact angle (α˜90 degree), as shown in FIGS. 1A to 5. Therefore, an end portion of the measuring sensor portion 30 is in good contact with a surface of the measured fruit F, and minimizes an interference of a hand of the user to the fruit F to improve the accuracy of the measurement.

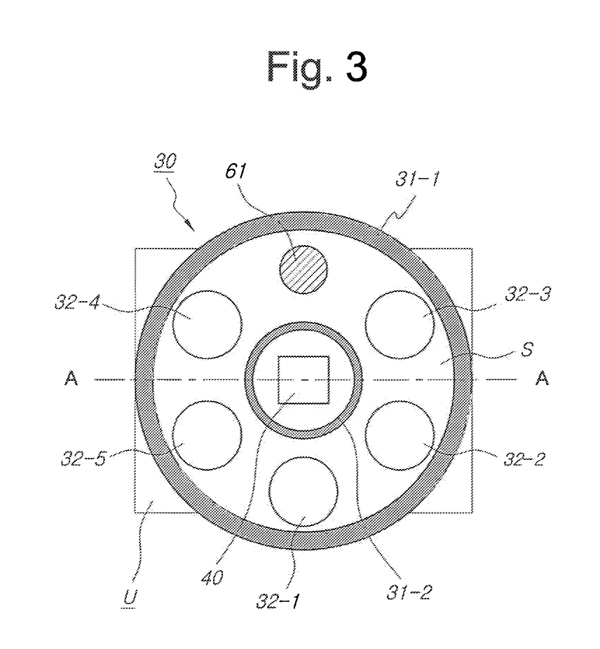

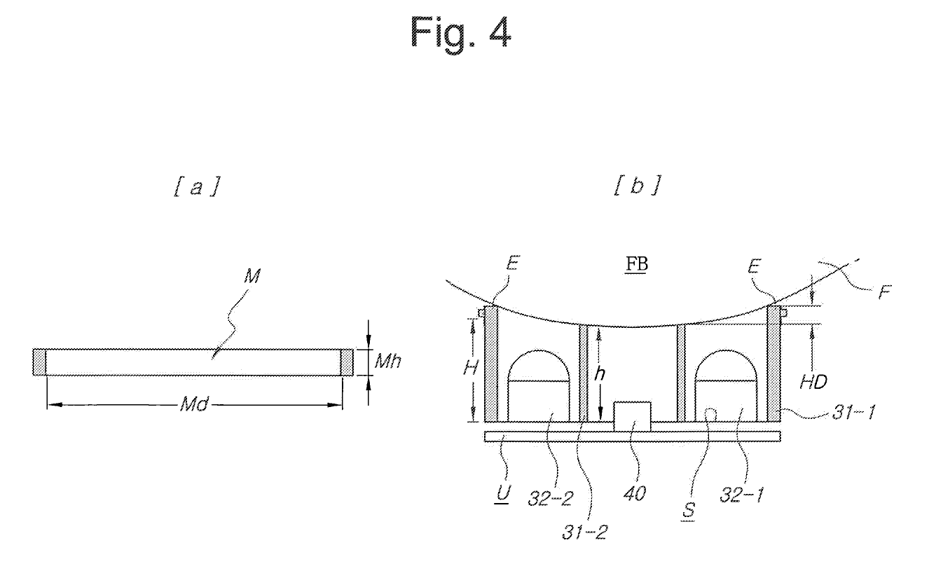

As shown in FIGS. 3 and 4B, the measuring sensor portion 30 has a light insolating tube 31-1 as an outer wheel tube which has a low height H, and a light department tube 31-2 as an inner wheel tube which has a diameter smaller than that of the light isolating tube 31-1 and extends from the rear surface of the casing in a coaxial direction of the light isolating tube 31-1, and which is disposed in the light isolating tube 31-1. It is preferred that the light department tube 31-2 has a height smaller than that of the light isolating tube 31-1.

As a result of testing fruits with lots of curvature R, a difference of the heights HD is preferably about 1 mm.

In the case where the non-destructive sugar content apparatus 100 is in contact with the fruit F as shown in FIG. 5 in order to describe the measuring process, since the fruit F such as an apple has a curvature R according to its coherent curved shape, a measured center portion of the apple is inserted at a desired depth into the measuring sensor portion 30 of the non-destructive measuring apparatus 100 due to the curvature R of the fruit F of the contact surface during the measurement by the non-destructive sugar content apparatus. The introducing depth is offset by the height difference between the light isolating tube 31-1 and the light department tube 31-2, so that the non-destructive sugar content measuring apparatus 100 is in close contact with the surface of the fruit. Accordingly, the reason for the difference height HD between the light isolating tube 31-1 and the light department tube 31-2 is to isolate a leakage of light from the LED light source 32 described later and an introduction of interference light from an exterior environment.

In order to achieve the above-mentioned object, the light isolating tube 31-1 of the measuring sensor portion 30 preferably is in contact with the surface of the fruit, which is a subject to be measured, without a scar on the fruit, resulting in an improvement of the measurement accuracy. Further, since the curvature R of the fruit F to be measured is different according to the kind of fruits F, the light isolating tube 31-1 is formed of an elastic material, preferably, rubber or urethane. More preferably, the light isolating tube 31-1 is made of a hard plastic in an injection molding method. It is possible to make a separate elastic material including rubber as a contact mount (M) with a sectional ring shape, as shown in FIG. 4A and to insert the contact mount (M) on an end (E) of the light isolating tube 31-1.

In the case of including the contact mount (M), a number of contact mounts (M) with different diameters (Md) and heights (Mh) are provided to the non-destructive sugar content measuring apparatus 100 of the present invention, so as to allow the non-destructive sugar content measuring apparatus 100 to be effectively used for various fruits with a small diameter and a large diameter.

The above-mentioned light isolating tube 31-1 isolates an introduction of unnecessary natural light or diffused natural light from an external environment to the fruit F and a point to be measured, and the light department tube 31-2 is a double tube and collects light flux at a point to be measured while preventing the dispersion of the light flux from the LED 32-1, 32-2, 32-3 as the LED light source 32 so that the light flux is introduced on the surface of the fruit F and sends the reflected light to the light sensor 40, resulting in the improvement of the measurement accuracy.

As shown in FIG. 4B, the plural LEDs 32-1, 32-2, 32-3, 32-4 and 32-5, which are the LED light source 32 for the measurement, are arranged in a circle on a bottom surface S of a ring shaped peripheral portion between the light isolating tube 31 and the light compartment tube 31-2 of the measuring sensor unit 30, and the light sensor 40, which receives light from the fruit F, is disposed at a center portion of the light isolating tube 31-1.

The light sensor 40 is a sensor arranged on a sensor unit U as described later, and is fixed to a whole sensor unit U as shown in FIG. 4B.

Further, a temperature sensor 61 may be disposed at a certain point among the LEDs 32-1, 32-2, 32-3, 32-4 and 32-5 which are the LED light source 32 and are arranged in a circle, so as to measure a temperature on a measured point at time point when the sugar content of the subject is measure. In the measuring process as described later, the temperature of the subject is employed as an important parameter.

In the present invention, the plural LEDs 32-1, 32-2, 32-3, 32-4 and 32-5 with a wavelength of 700-900 nm are selected as the suitable LED light source 32.

As shown in FIGS. 3 and 4B, the measuring sensor portion 30 is constructed in such a manner that the LEDs 32-1, 32-2, 32-3, 32-4 and 32-5, which are the light source, and the light sensor 40, which is the light receiving portion, are optically compartmented and designed so that the light of the light source, which is not passed through the surface of the fruit F, is prevented from being introduced into the light sensor 40. Therefore, the LED with relatively low brightness can be used as the light source and it is possible to reduce a manufacturing cost and a consumption of electric power in comparison with the structure in which the conventional light source with high brightness has been used. Accordingly, it is possible to develop the measuring sensor portion 30 which is suitable for a portable device.

As shown in FIG. 5, light I projected toward the fruit F includes regularly reflected light Ir which is reflected by the surface of the fruit F, reflected light Ib which permeates in and passes through flesh FB of the fruit F, and is transmitted to the surface of the fruit F, diffused light Id which permeates from the external environment, absorbed light Ia which is absorbed by the flesh FB in the fruit F, and penetrating light It which penetrates the flesh FB.

Accordingly, the light isolating tube 31 and the light compartmenting tube 31-2 of the measuring sensor unit 30 minimize the diffused light Id which permeates in the fruit from the external environment and transmits only the reflected light Ib, which permeates in the fruit F and passes through the flesh FB, and is transmitted to the surface of the fruit F, to the light sensor 40, thereby improving the accuracy in the measurement of sugar content. The light flux is collected from the light source and maximally used for the measurement of the sugar content, thereby making it possible to use the LED light source with relatively low brightness. Therefore, there is an advantage in that problems such as complexity, maximization, and high cost of the structure due to a use of a halogen lamp of high brightness of the conventional application can be solved.

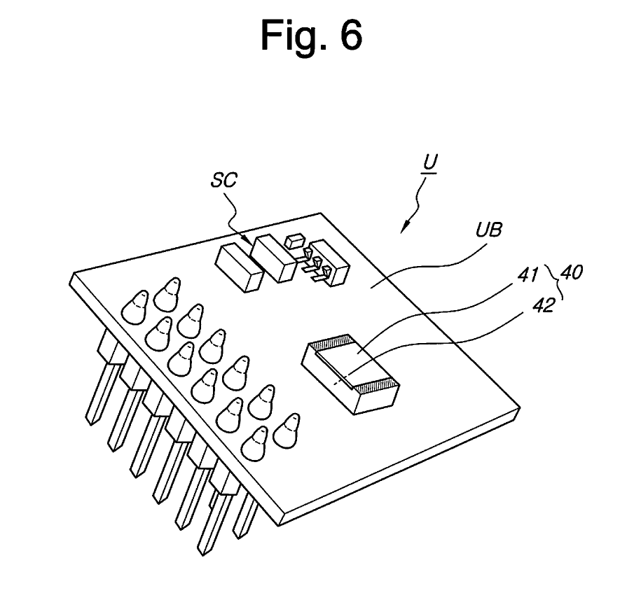

The light sensor 40 according to the present invention employed to the light sensor unit 30 is configured to be used as a sensor, and to be mounted as an element on the sensor unit U of a circuit board UB separately from the main printed circuit board PB, as shown in FIG. 6.

In the sectional view of the measuring sensor unit 30 shown in FIG. 4B, the optical sensor 40 is mounted on a bottom surface of the light compartment tube 31-2 which is arranged at a center portion of the measuring sensor unit 30, and exposed outwardly. A cover glass (not shown) is preferably mounted at an exposed side of the light sensor 40.

The optical sensor 40 is a Near Infrared Ray (NIR) spectral sensor according to the convention art, and has been used for the various purposes up to now.

The NIR spectral sensor according to the conventional art is disclosed in Korean Patent aid-Open Publication No. 10-2011-0111970, entitled “Integrated light sensor and method for measuring sugar content of fruit”. Since the NIR spectral sensor is constructed by integrating and arranging a Light Emitting Diode, and a photodiode and a sensor for detecting light reflected by a tissue of a fruit, on a substrate, or assembled by separately making a filter array on a quartz or glass wafer, which is made in a pre-process and attaching the filter array on a chip, in which a CMOS Image Sensor (CSI) is additionally integrated, in a hybrid form, there is a problem in that a price of the NIR spectral sensor is very expensive. Further, since the NIR spectral sensor is not produced in large quantities but is a semi-product in which existing parts are assembled, there are problems in that a yield is low, a manufacturing cost increases due to multi-stage processes, and operational reliability is low.

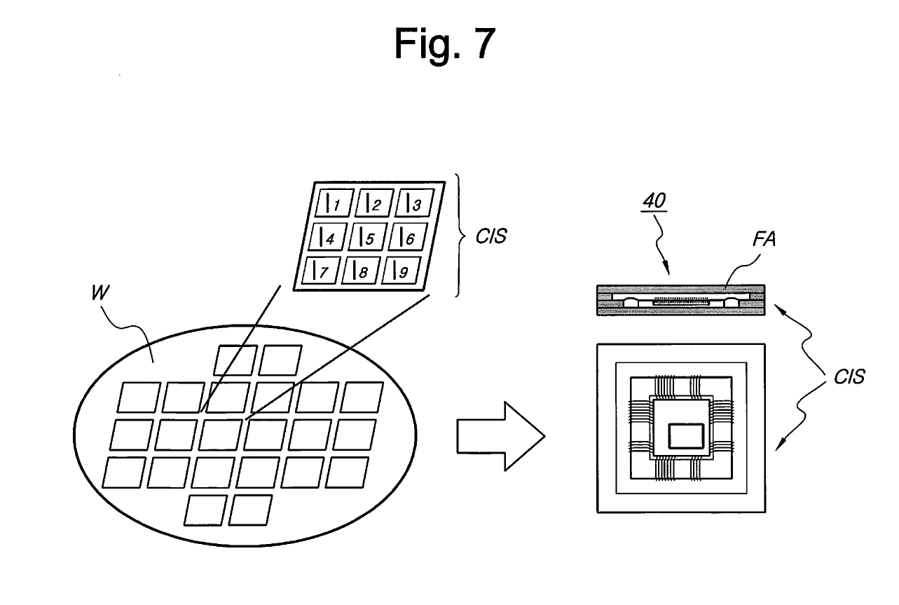

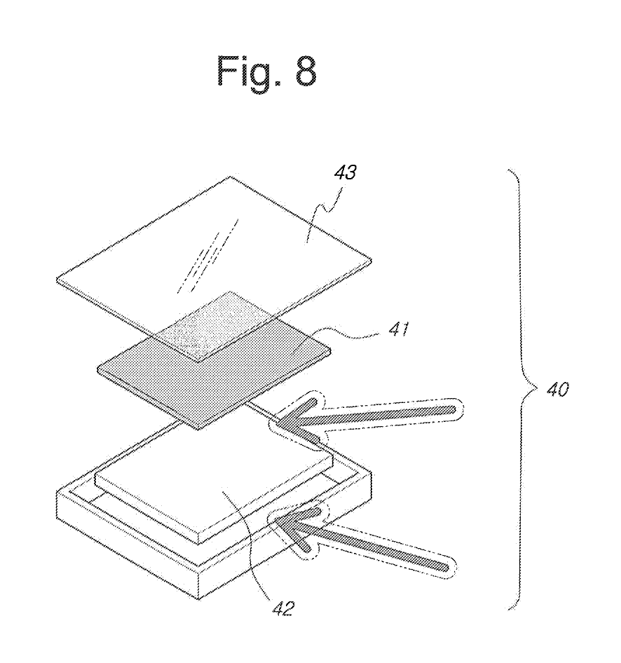

The optical sensor 40 arranged on the sensor unit U of the measuring sensor portion 30 which is employed to the present invention is manufactured in such a manner that nano-filter arrays with a spectral characteristic of 700 nm˜900 nm are made on the CIS wafer by using a semiconductor process of optical lithography and integrated monolithically, as shown in FIGS. 7 and 8. Accordingly, the optical sensor 40 allows the non-destructive sugar content measuring apparatus to be easily carried in comparison with the conventional products, which is disclosed in the preceding application and is incorporated in the present invention for reference.

As shown in FIG. 7, the light sensor 40 is manufactured by arranging and matching nano-filter arrays FA with pixels of the CIS on the CIS wafer by using the semiconductor process of the optical-lithography through an integrated process.

The structure of the optical sensor 40 is already disclosed in the preceding application, and is adapted to the present invention. Thereby, the price of the sensor unit, which is a significant portion of the price of the non-destructive sugar content apparatus 100, can be lowered.

In the optical sensor 40, a spectral filter 41 which is the nano-filter array of FIG. 8, and manufactured in the above-mentioned method, filters a certain waveband, for example a waveband of 750 nm, and the CIS 42 converts a wavelength of a filtered light into an electric signal. It will be understood that a cover glass 43 may be mounted on the optical sensor 40 in order to protect the optical sensor.

The optical sensor 40 constructed as described above is fixed as a light receiving body to the sensor unit U made in the form of the sensor circuit substrate UB which is a separate circuit substrate as shown in FIG. 7, and control elements are arranged on the sensor unit U in order to process optical signals as described later. One of the control elements may be a sensed signal processing microcomputer SC such as a standardized custom integrated circuit, and has logic for processing signals, as described later.

Of course, a position of the main circuit substrate PB in the casing is matched with a position of the measuring sensor portion 30, and the separate sensor circuit substrate UB of the sensor unit U can be removed. However, it is preferred to separately construct the main circuit substrate PB and the measuring sensor portion 30 as separate substrates in view of a management of producing and manufacturing processes.

The optical sensor 40 is the NIR spectral sensor unit and used as an element for detecting optical data sensed and filtered from a sample subject because of using an intensity of a wavelength of light. The optical sensor 40 is arranged on the sensor unit U and operates as the control element.

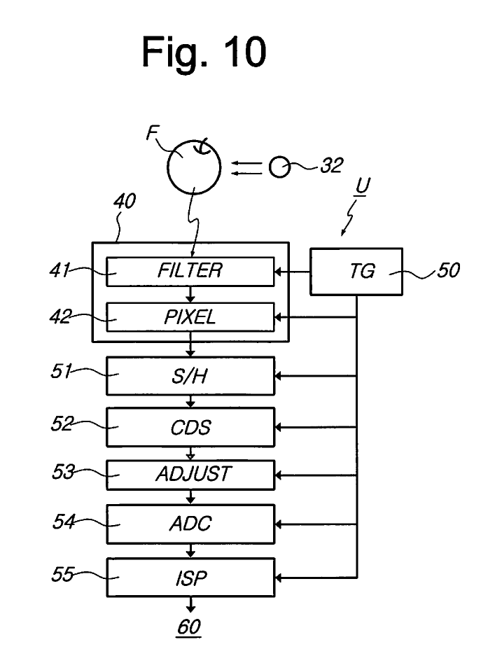

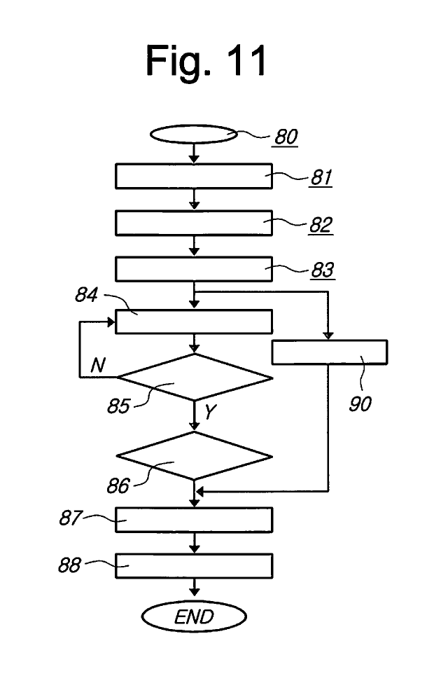

An electric signal process operating along with the logic of processing signal in the sensed signal processing microcomputer SC on the sensor unit U which is incorporated with the optical sensor 40 will be described with reference to FIG. 10.

[Signal Processing of the Sensor Unit U and the Light Sensor 40]

The sensed signal processing microcomputer SC of the control element on the sensor unit U which is incorporated with the light sensor 40 is provided with signal processing blocks as described below.

Pixel arrays on the CIS 42 are driven along a driving pulse from a transfer gate 50, and output light from the plural LEDs 32-1, 32-2, 32-3, 32-4 and 32-5 which are the LED light source 32 is introduced into the fruit F by a cooperation of the light isolating tube 31-1 and the light compartmenting tube 31-2, and the reflected light Ib, which penetrates through the flesh of the fruit F and is reflected toward the surface of the fruit, is filtered by the spectral filter 41 which is the nano-filter array of the optical sensor 40 so that a certain waveband of the near infrared ray, for example a waveband of 750 nm, remains. The remaining NIR waveband light drives the pixel arrays on the CIS 42, so as to obtain electric signals based on the reflected NIR wavelength.

A sampling and holding portion 51 performs and stores a sampling of a signal of driving the pixel arrays on the CIS 42 according to the driving pulse from the transfer gate 50, based on the electric signal, and a Correlated Double Device (CDD) 52 performs a sampling of signals before and after receiving the signal form the CIS 42. In turn, the sampling and holding portion 51 removes noise generated before receiving the signal and unique non-uniformity of the pixels of the CIS 42 by subtracting each other and an adjusting portion 53 adjusts a gain and corrects an offset value. Then, an Analog to Digital Converter (ADC) 54 converts an electrical analog signal into a digital signal, and an Image Sensing Processor (ISP) 55 processes an image signal converted into the digital value so as to generate an electric signal of a more accurate image data, and transmits the electric signal to a Central Processing Unit (CPU) 60 of the main microcomputer of the main circuit board PB so that the CPU 60 processes the electric signal.

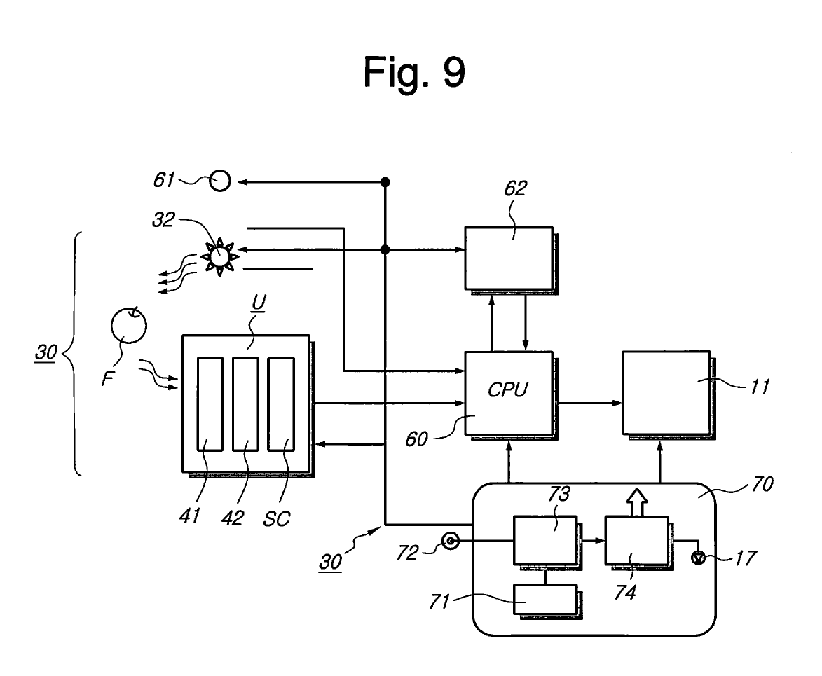

The structure of the main circuit board PB cooperated with the sensor unit U will be described with reference to FIG. 9.

The CPU 60 is mounted on the main circuit board PB which is a PCB embedded in the casing, which processes, calculates and determines the electric signal from the sensor unit U. The CPU 60 has a control logic embedded therein in the program form.

The CPU 60 has a temperature sensor 61 and includes a converting logic according to algorithm of converting the electric signal from the sensor unit U into a numeric value because a temperature value of a measured environment at the time of measuring the sugar content is necessary as a correction data.

A temperature data value from the temperature sensor 61 and a light source data value from the LED light source 32 are transmitted to the CPU 60 through an EPROM 62 in which the temperature data value and the light source data value are stored for a calculation, and an output value from the CPU 60 is displayed on the digital display 11 such as an LCD of the front half body 1.

A supply of electric power to the main circuit board PB is performed by the electric power source portion 70. The electric power source portion 70 is provided with a rechargeable battery 71 such as a general lithium ion battery and is charged by an external electric power source 72 so as to supply electric power to the main circuit board PB through a battery controller 73 and a DC/DC converter 74. It is preferred that the non-destructive sugar content measuring apparatus is provided with a chargeable electric power supplying means because it is repeatedly and continuously used.

The DC/DC converter 74 supplies electric power to the temperature sensor 61, the LED light source 32, the EPROM 62, the sensor unit U, the CPU 60 and the digital display 11 which are structural elements of the non-destructive sugar content measuring apparatus 100 after converting a voltage of the electric power into a voltage suitable for a level required to the structural elements.

A charging state of the rechargeable battery is displayed on a charging state displaying portion 11-4, and via an electric power displaying light 17 and a charging state light 18 on the panel portion 10.

Many theses and documents published before the present invention was filed disclose an algorithm and a program capable of analyzing an optical waveform which is measured, based on a wavelength data which is an electric processing signal from the sensor unit U, a temperature data from the temperature sensor 61 and a light source data from the LED light source 32 which are received in the CPU 60 on the main circuit board PB, and calculating the sugar content based on a calibration method of an optical data conversion and a fitting method. In the present invention, one of the theses and the documents suitable for the present invention is exemplary described.

The mode selection switch 13, the up and down switches 14 and 15 for the selection of menus, and the OK switch 16 for the confirmation, which are arranged on the panel portion 10, are not essential structural elements of the present invention. However, they are necessary operational switches in the case where the CPU 60 selects a multiple regression analysis value with different constants and parameters, which are suitable for a various kinds of fruits according to a statistical data.

[A Signal Processing of the Main Circuit Board PB and the CPU 60 of the Non-Destructive Sugar Content Apparatus 100]

Based on the electric signal which is processed by the light sensor 40 of the sensor unit U, when the measurement of sugar content is started by an operation of the measurement starting button 21 in a state that the measuring sensor portion 30 is in contact with the fruit F to be measured as shown in FIG. 4B, in step 80, a shut speed of the optical sensor 40 is selected in step 81, and a back data which is a previous measured data is measured in step 82 (the back data is measured in a state that the LED is not turned on) and is reset to ‘0’ in step 83.

In an initial state, the LED light source 32 is turned on and emits light into the fruit F to enable the light to react with sugar in the flesh FB, and then the optical sensor 40 on the sensor unit U detects a near infrared ray spectrum of the reflected light Ib which is reflected by the flesh FB and converts the near infrared ray spectrum of the reflected light into electric signals in step 84. Continuously, the sensor unit U processes the electric signals before transmitting the electric signals to the CPU 60 on the main circuit board PB.

The temperature sensor 61 measures a temperature of the fruit F at a measuring time in step 90.

It is determined whether a value that the light sensor 40 measures of an internal reflective wave is in a range of a reference value (i.e. a range of valid data or invalid data which has a very large size or a very small size) in step 85. If the measured value of the optical sensor 40 is not in a range of the reference value, the optical sensor measures the internal reflective wave again.

The CPU 60 repeats these measuring processes several times, and performs a measurement calibration step if the measured data is in a range of the reference value in step 86. When the measurement calibration is completed in the measurement calibration step, a temperature compensation for a measured temperature of the temperature sensor 61 is performed in step 87. Then, the CPU 60 processes the measured data, enables the digital display 11 to display sugar content in step 88, and terminates the measurement of the sugar content.

The measurement calibration in the measurement calibrating step 88 is carried out by using a generally known analysis method, for example, the multiple regression analysis disclosed in the Korean Patent Laid-Open Publication No. 10-2011-0111970. Through the analysis method, an experiment is repeatedly carried out by substituting constants and object parameters for a basic measurement to obtain multiple regression values.

That is, the multiple regression analysis is well known as a regression analysis method to obtain a relational expression between Y and X1, X2, . . . , Xn, in which an object parameter Y is expressed as a linear equation of description parameters X1, X2, X3, . . . , Xn which are sugar contents measured by the numbers n of the optical sensors 40, i.e. Y=a+b1X1+b2X2+b3X3+, . . . +bnXn.

Here, a is a constant, and X1, X2, X3, . . . , Xn are sugar contents measured by the optical sensor 40. Also, b1, b2, b3, . . . , bn are regression coefficients. Values of sugar contents are compensated according to the measured temperature values and then a brix value is displayed on the digital display 11.

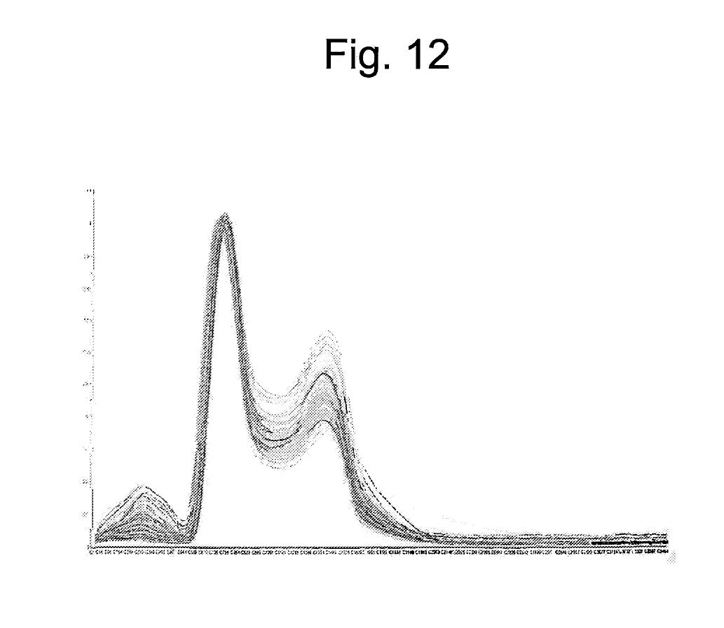

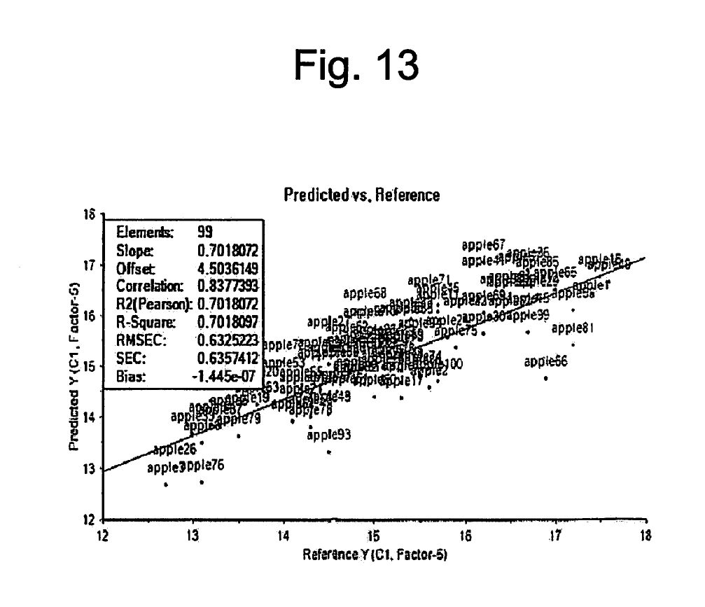

The constant a and the sugar contents b1, b2, b3, . . . , bn are obtained through the experiment, which are values obtained by statistically processing experiment values of spectrums of measuring light for different sugar contents. FIG. 12 is a graph illustrating spectrums of apples according to their sugar contents, for which the experiment of the present invention is repeatedly carried out to obtain, and FIG. 13 is a graph illustrating a correlation between an actual sugar content of the fruit F and sugar content as a value which is measured by the optical sensor 40 and obtained by performing a calibration for light to be measured, in which the calibration is carried out in order to establish a sugar content prediction model for an apple.

### INDUSTRIAL APPLICABILITY

As described above, the non-destructive sugar content measuring apparatus of the present invention allows a user to simply operate a light emission and to identify accurate sugar content as a numeric value without a destruction of fruits during a sampling or total inspection of fruits. Accordingly, the user can determine an optimized forwarding and selling time, resulting in an improvement of agricultural productivity and a sale of optimized products in a distribution process.

What is claimed is:

1. A non-destructive sugar content measuring apparatus, comprising:
a measuring sensor portion 30 for including an LED light source 32 which has LEDs 32-1, 32-2, 32-3, 32-4 and 32-5 circularly arranged, an optical sensor 40 which receives light reflected by a flesh of a fruit F, and a temperature sensor 61, in order to predict sugar content through a statistical analysis method in which the optical sensor 40 to be used as a spectral sensor receives a near infrared ray from the light which is emitted from the light source 32 and reflected by the flesh FB of the fruit F of which the sugar content is measured;
a casing including a rear half body 20 on which the measuring sensor portion 30 extends outwardly and a front half body 1 combined with the rear half body 20 to constitute the casing of the sugar content measuring apparatus 100, the front half body 1 having a panel portion 10 which has a digital display 11 for displaying a brix value as a digital value and operational switches, the panel portion and the operational switches being mounted on a front face thereof;
a main circuit board PB for including a Central Processing Unit (CPU) 60 which processes electric signals from the optical sensor 40 and performs a calculation and determination, an EPROM 62 which stores temperature data from the temperature sensor 61 and light source data from the LED light source 32, and a rechargeable electric power supplying portion 70, the main circuit board being embedded in the casing; and
the CPU 60 enabling the digital display 11 to display the sugar content as a numeric value which is obtained by processing a light waveform, which is measured based on wavelength data corresponding to electric processing signals from the optical sensor 40, temperature data from the temperature sensor 61 and light source data from the LED light source 32, by means of a statistic analysis method,
wherein the optical sensor 40 is an element on the sensor unit U which is mounted on a circuit board UB separated from the main circuit board PB, and has a spectral filter 41 and a CMOS Image Sensor (CIS) 42 in which nano-filter arrays are mass-manufactured and monolithically integrated on a CIS wafer by using a semiconductor process of an optical lithography, a sensor signal processing microcomputer SC is mounted, as a pretreatment means for processing and transmitting an electric signal from the optical sensor 40 to the CPU 60, on an identical circuit board as that for the optical sensor 40, and constitutes an independent sensor unit U, and the optical sensor 40 is disposed in the measuring sensor portion 30 and exposed outwardly.

2. The non-destructive sugar content measuring apparatus as claimed in claim 1, wherein the sensor signal processing microcomputer SC, which is mounted on the sensor unit U and is cooperated with the optical sensor 40, further comprises:
a transfer gate 50 for driving pixel arrays on the CIS 42 according to a generated driving pulse;
a sampling and holding portion 51 for sampling and temporarily storing a signal from the optical sensor 40, which is caused by reflected light of the fruit F;
a correlated double device 52 for removing a noise and a coherent unevenness of pixels of the CIS, 42 which are generated prior to a reception of the signal, by sampling a signal twice and subtracting the signals from each other before and after receiving the signals from the CIS 42;
an adjustment portion 53 for adjusting a gain of the processed signal and compensating an offset value;
an analog-digital converter 54 for converting the processed analog signal into a digital signal; and
an Image Sensor Processor (ISP) 55 for processing an image signal converted into the digital value so as to make an electric signal of an image data more accurately and to transmit the electric signal to the CPU 60 which is the main microcomputer of the main circuit board PB.
